(19) United States
(12) Patent Application Publication
(10) Pub. No.: US 2019/0201314 A1
(43) Pub. Date: Jul. 4, 2019

(54) SKIN PERFECTING COSMETIC COMPOSITIONS AND METHODS OF USE

(71) Applicant: L'OREAL, Paris (FR)

(72) Inventors: Alexandra Jane Elisa FARRAN, Dayton, NJ (US); Anne-Laure Suzanne BERNARD, New-York, NY (US); Erin MCMULLIN, Ewing, NJ (US)

(21) Appl. No.: 16/234,738

(22) Filed: Dec. 28, 2018

Related U.S. Application Data
(60) Provisional application No. 62/611,703, filed on Dec. 29, 2017.

(51) Int. Cl. A61K 8/87; A61K 8/02; A61K 8/81; A61K 8/25; A61K 8/891; A61K 8/31; A61K 8/34; A61K 8/86; A61Q 19/08

(52) U.S. Cl. CPC A61K 8/87; A61K 8/0204; A61K 8/8152; A61K 8/25; A61K 8/891; A61K 2800/48; A61K 8/31; A61K 8/34; A61K 8/345; A61K 8/86; A61Q 19/08; A61K 8/8158

(57) ABSTRACT

The instant disclosure relates to cosmetic compositions that when applied to skin instantaneously and dramatically improve the appearance of the skin. The compositions include at least one water-dispersible primary film forming polymer having a Young's modulus of 0.1 to 50 MPa, an elongation at break >50%, and a glass transition temperature of <0° C.; optionally, at least one secondary, non-polyurethane film forming polymer having a glass transition temperature of <25° C.; optionally, at least one pressure sensitive adhesive polymer; soft focus powders; and water. Methods of using the cosmetic compositions, for example, methods for improving the appearance, are also disclosed.

### CROSS-REFERENCE TO RELATED APPLICATIONS

This application claims priority to U.S. Provisional Patent Application No. 62/611,703, filed on Dec, 29, 2017, entitled “SKIN PERFECTING COSMETIC COMPOSITIONS AND METHODS OF USE,” the entirety of which is herein incorporated by reference.

### FIELD OF THE DISCLOSURE

The present disclosure relates to cosmetic compositions for application to the skin for providing an instantaneous and dramatic improvement to the appearance of skin, for example, by reducing the appearance of wrinkles, eye bags, pores, and skin imperfections such as scarring, dark spots (and uneven skin tone), dark circles, and roughness.

### BACKGROUND

Skin produces less collagen and elastin as it ages. For example, after the age of twenty, a person (human) produces about 1 percent less collagen in the skin each year. As a result, the skin becomes thinner and more fragile. Inevitably, wrinkles, crow's feet, age-spots, eye bags, and the like, begin to form.

Consumers often wish to improve the appearance of such age-related skin imperfections, preferably with instantaneous results. Many consumer products and procedures devoted to hiding and reducing wrinkles are available. Some products and procedures are simple and inexpensive, for example, applying make-up, particularly a primer or colored foundation, to cover the skin (and thereby cover and/or fill the wrinkles and provide a smoother look). Far more expensive and drastic procedures, such as surgical face lifts and Botox® injections, are also used to reduce the appearance of wrinkles. However, many consumers either cannot afford, or do not wish, to undergo such drastic cosmetic procedures. There are a number of lotions and creams which are formulated to hydrate the skin and make it more supple, thereby reducing the appearance of wrinkles. Some of these products contain active ingredients, for example, niacinamide, that help repair and rejuvenate skin over time. Unfortunately, however, all of these products and procedures have drawbacks.

Make-up products are often visible, offer minimal texture benefits, and have no long-term lasting effect on the skin. After removal of the make-up, the skin looks the same as before the make-up was applied. Common skin care products can have chronic, acute or both effects on the skin. Hydration and optical effects are common acute benefits, but these benefits quickly wear-off over time.

Attempts have been made to develop new categories of products to improve the appearance of skin without the drawbacks of existing products and procedures. One such family of products can be generally classified as “adhesive, contractile film formers”. Film formers are chemical compositions that when applied to skin, leave a pliable, cohesive and continuous covering. A select group of film formers are also adhesive to the skin and contractile.

### SUMMARY OF THE DISCLOSURE

The instant disclosure relates to skin perfecting cosmetic compositions, i.e., cosmetic compositions that are applied to the skin and improve the appearance of the skin. The compositions include a combination of ingredients such as film-forming polymers, a pressure sensitive adhesive, and soft focus powder, which combine to form a composition that instantaneously and dramatically improves the appearance of skin. For example, upon application to the skin, the compositions reduce the appearance of wrinkles, eye bags, pores, and skin imperfections such as scarring, dark spots (and uneven skin tone), dark circles (typically around the eyes), and roughness. Unlike other products, the films formed on the skin do not dry-out, whiten, crack, or peel. Instead, they remain flexible (elastic), durable, and comfortable. Moreover, the compositions (and resulting films) hydrate and protect the underlying skin.

The cosmetic compositions of the instant disclosure typically include:
at least one primary water-dispersible film forming polymer having a Young's modulus of 0.1 to 50 MPa, an elongation at break >50%, and a glass transition temperature of <0° C.;
optionally, at least one secondary, non-polyurethane water-dispersible film forming polymer having a glass transition temperature of <25° C.,
optionally, at least one pressure sensitive adhesive polymer;
soft focus powder;
at least one thickening agent;
optionally, at least one water soluble solvent; and
water.

In many instances, additional ingredients are also present in the cosmetic compositions. Non-limiting examples of additional ingredients that may be present include preservatives, coalescents, wetting agents, emollients, etc.

In some cases, the glass transition temperature of the at least one primary water-dispersible film forming polymer and the at least one secondary film forming polymer, if present, differs by at least 20° C.

In some instances, the at least one primary water-dispersible film forming polymer is a polyurethane latex polymer, for example, those that are formed by reacting a di- or polyisocyanate with a diol and/or polyol, including for example, aqueous polyurethane dispersions. Non-limiting examples include polyurethane 32, polyurethane-34, polyurethane-35, polyurethane-48, and a mixture thereof. In some cases, the cosmetic compositions preferably include at least two primary water-dispersible film forming polymers having a Young's modulus of 0.1 to 50 MPa, and elongation at break >50% and a glass transition temperature of <0° C., for example, polyurethane-34 and polyurethane-35.

In some instances, the at least one secondary non-polyurethane water-dispersible film forming polymer is an acrylate polymer. Non-limiting examples of acrylate polymers include acrylates copolymer, styrene/acrylates copolymer, acarylates/ethylhexyl acrylate copolymer, an alkyl acrylate copolymer, acrylic copolymers, polyacrylate-2 crosspolymer, acrylates/hydroxyesters acrylate copolymer, acrylate/ethylhexyl acrylate copolymer, styrene acrylate copolymer, acrylate/VA copolymer, styrene/acrylic copolymer, styrene/acrylates copolymer, styrene/acrylates/ammonium methacrylate copolymer, and a mixture thereof.

The total amount of all film forming polymers in the cosmetic composition may vary but in some cases is at least 10 wt. %, for example, at least 10 wt. % to about 40 wt. %, based on the total weight of the cosmetic composition. Additionally, the total amount of primary film forming polymers is typically higher than the total amount of secondary film-forming polymers (if present), for example, the composition may include at least double the total amount of primary film forming polymers than the total amount of secondary film forming polymers.

Pressure sensitive adhesive polymers are not required. Nonetheless, in many instances, inclusion of a pressure sensitive adhesive polymer is useful. Non-limiting examples of pressure sensitive adhesive polymers include copolymers of acrylate and methacrylate, rubber-based polymers, acrylic polymer, and styrene copolymers (such as styrene-isoprene-styrene (SIS) and styrene-butadiene-styrene (SBS) copolymers). In some instances, the pressure sensitive adhesive polymer is an acrylate polymer, for example, a copolymer of butyl acrylate, butyl methacrylate or acrylic acid. A non-limiting examples of such polymer is 2-ethylhexylacrylate (also referred to as (poly)ethylhexylacrylate).

Soft focus powders (or gels) are useful for providing blurring effects, which provides skin with a smoother appearance, for example, by reducing the difference in luminosity between the valley and the edges of wrinkles and imperfections. For example, soft focus powders may be powders of natural or synthetic origin such as mica, titanated mica, alumina, aluminum silicate, silica which may or may not be coated, fumed silica, silica silylate, titanium dioxide, serecite, composite talc/titanium dioxide/alumina/silica powders, polyamide, poly(methyl (meth)acrylate), polyethylene powder, polymethylsilsesquioxane powder, waxes, such as copernicia cerifera (carnauba) wax, dimethicone/vinyl dimethicone crosspolymer, nylon-12, cellulose, polylactic acid, boron nitride, and a mixture thereof. The copernicia cerifera (carnauba) wax can be provided as a dispersion in water and alcohol. The dimethicone/vinyl dimethicone crosspolymer can be provided as silicone dispersion (INCI: Dimethicone/vinyl dimethicone crosspolymer (and) C12-14 Pareth-12).

The at least one thickening agent may be a hydrophilic thickening agent. Non-limiting examples include modified or unmodified carboxyvinyl polymers, acrylates/C10-30 alkyl acrylate crosspolymer, polyacrylates, polymethacrylates, polyacrylamides, optionally crosslinked and/or neutralized 2-acrylamido-2-methylpropane sulfonic acid polymers, crosslinked anionic copolymers of acrylamide and of 2-acrylamido-2-methylpropane sulfonic acid (AMPS) (e.g., acrylamide/sodium acryloyldimethyltaurate copolymer), polysaccharides, celluloses, and a mixture thereof.

The instant disclosure also relates to methods for improving the appearance of skin comprising applying the compositions described herein to the skin. In some instances, the compositions are applied to the skin of the face and/or neck and may be specifically applied around the eyes, around the mouth, and/or around the neck of a human face. The methods of improving the appearance of skin include methods for treating or reducing the appearance of wrinkles, blemishes, dryness, roughness, dullness, age spots, discoloration, scarring, sagging, and/or puffy skin.

### DETAILED DESCRIPTION OF THE DISCLOSURE

The present disclosure relates to skin perfecting cosmetic compositions and methods for improving the appearance of skin using the compositions. The cosmetic compositions include a unique combination of film forming polymers and at least one pressure sensitive adhesive polymer, which provides tackiness to the composition. The unique combination of film forming polymers and pressure sensitive adhesive polymer(s) interact such that the cosmetic compositions, upon application to skin, provide an instantaneous and dramatic improvement to the appearance of the skin, for example, by reducing the appearance of wrinkles, eye bags, pores, and skin imperfections such as scarring, dark spots (and uneven skin tone), dark circles (typically around the eyes), roughness, etc.

The cosmetic compositions include at least one, and preferably at least two, primary water-dispersible film forming polymers having a Young's modulus of 0.1 to 50 MPa, an elongation at break >50%, and a glass transition temperature of <0° C. In some instances, the cosmetic compositions include at least one, and preferably at least two, primary water-dispersible film forming polymers having a Young's modulus of 0.1 to 30 MPa, an elongation at break >50%, and a glass transition temperature of <0° C. These polymers provide a tightening effect to the skin yet retain elasticity and therefore are able to follow facial movements without cracking and peeling.

At least one secondary, non-polyurethane film forming polymer may also optionally be included in the cosmetic compositions. The optional, secondary, non-polyurethane film forming polymers typically have a glass transition temperature of <25° C. Furthermore, the glass transition of the at least one primary water-dispersible film forming polymer and the at least one secondary film forming polymer, if present, typically differs by at least 20° C. The glass transition of the at least one primary water-dispersible film forming polymer and the at least one secondary film forming polymer, if present, may differ by at least 30° C., 40° C., 50° C., 60° C., 70° C., or 80° C. A maximum difference between glass transition temperatures may be 50° C., 60° C., 70° C., 80° C. or 100° C.

In addition to its film forming properties, the secondary, non-polyurethane water-dispersible film forming polymer can be helpful in providing a variety of other beneficial properties to the cosmetic compositions, for example, adhesiveness, texture, and durability. Finally, the cosmetic compositions include at least one pressure sensitive adhesive polymer. Pressure sensitive adhesive polymers differ from the primary water-dispersible and secondary non-polyurethane water-dispersible film forming polymers because they do not provide extensive film forming properties. Instead, their primary role is to act as an adhesive that binds or hold the cosmetic compositions to the skin.

In addition to the film forming polymers and the pressure sensitive adhesive polymer(s), the cosmetic compositions may additionally include soft focus powders, thickening agents, water, water-soluble solvents, pigments, preservatives, chelating agents, etc. More specifically, the cosmetic compositions of the instant disclosure may include at least the following:
at least one, and preferably at least two primary water-dispersible film forming polymers having a Young's modulus of 0.1 to 50 MPa (or a Young's modulus of 0.1 to 30 mPA), an elongation at break >50%, and a glass transition temperature of <0° C.;
optionally, at least one secondary, non-polyurethane water-dispersible film forming polymer having a glass transition temperature of <25° C.,
   wherein the glass transition of the at least one primary water-dispersible film forming polymer and the at least one secondary film forming polymer, if present, differs by at least 20° C.;
optionally, at least one pressure sensitive adhesive polymer;
soft focus powder;
at least one thickening agent;
optionally, at least one water soluble solvent; and
water.

Primary Water-Dispersible Film Forming Polymer

As noted above, the at least one primary film forming polymer has a Young's modulus of 0.1 to 50 MPa, an elongation at break >50%, and a glass transition temperature of <0° C. The Young's modulus may be lower, for example, from 0.1 to about 40 MPa, from 0.1 to 35 MPa, or 0.1 to 30 MPa. The elongation at break can be even higher, for example, >50%, >75%, or >100%. Also, the glass transition temperature may be lower, for example, <−5° C., <−10° C., <−15° C., <−20° C., or <−25° C. In some instances it is preferable to include at least two primary film forming polymers. For example a first primary film forming polymer may have a Young's modulus of 0.1 to 10 MPa and a second primary film forming polymer may have a Young's modulus of greater than 10 MPa to about 50 MPa, greater than 10 MPa to about 40 MPa, or greater than 10 MPa to about 30 MPa.

Non-limiting examples of useful primary film forming polymers include polyurethane latex polymers, for example, those that are formed by reacting a di- or polyisocyanate with a diol and/or polyol, including for example, aqueous polyurethane dispersions. More specific, non-limiting examples include polyurethane-32, polyurethane-34, polyurethane-35, polyurethane-48, and a mixture thereof. In one embodiment, the cosmetic compositions include at least two primary film forming polymers selected from the group consisting of polyurethane-32, polyurethane-34, polyurethane-35, polyurethane-48, and a mixture thereof, for example polyurethane-34 and polyurethane-35.

The total amount of primary film forming polymers in the cosmetic composition can vary but is typically about 1 to about 40 wt. %, based on the total weight of the cosmetic composition. In some cases, the total amount of primary film forming polymers is about 1 to about 35 wt. %, about 1 to about 30 wt. %, about 5 to about 40 wt. %, about 5 to about 35 wt. %, about 5 to about 30 wt. %, about 10 to about 40 wt. %, about 10 to about 35 wt. %, or about 10 to about 30 wt. %, based on the total weight of the cosmetic composition.

In instances where the cosmetic composition includes at least two primary film forming polymers (a first primary film forming polymer and a second primary film forming polymer), the first primary film forming polymer may have a Young's modulus of 0.1 to 10 MPa (and an elongation at break >50% and a glass transition temperature of <0° C.). The total amount of this first primary film forming polymer may be about 1 to about 25 wt. %, about 1 to about 20 wt. %, about 1 to about 15 wt. %, about 5 to about 25 wt. %, about 5 to about 20 wt. %, or about 5 to about 15, based on the total weight of the cosmetic composition. The second primary film forming polymer may have a Young's modulus of greater than 10 MPa to about 50 mPa (and an elongation at break >50% and a glass transition temperature of <0° C.). The total amount of the second primary film forming polymer may be about 1 to about 25 wt. %, about 1 to about 20 wt. %, about 1 to about 15 wt. %, about 5 to about 25 wt. %, about 5 to about 20 wt. %, or about 5 to about 15, based on the total weight of the cosmetic composition. The weight ratio of the first primary film forming polymer (having a Young's modulus of 0.1 to 10 MPa) to the second primary film forming polymer (having a Young's modulus of greater than 10 MPa to about 50 mPa) may be about 1:5 to about 5:1, about 1:3 to about 3:1, about 1:2 to about 2:1, about 1.5:1 to about 1:1.5, or about 1:1.

Secondary, Non-Polyurethane Water-Dispersible Film Forming Polymer

The at least one secondary, non-polyurethane water-dispersible film forming polymer has a glass transition temperature of <25° C. Non-limiting examples of film forming polymers include acrylic polymers or co-polymers, acrylates, polyolefins, polyvinyls, polacrylates, silicones, polyamides, polyethers, polyesters, fluoropolymers, polyethers, polyacetates, polycarbonates, polyamides, polyimides, rubbers, epoxies, formaldehyde resins, organosiloxanes, dimethicones, methicones, cellulosics, polysaccharides, polyquaterniums, and the like. Suitable film formers include those listed in the Cosmetic Ingredient Dictionary (INCI and Handbook, 12^{th} Edition (2008), the disclosure of which is hereby incorporated by reference.

In some instance, the at least one secondary, non-polyurethane water-dispersible film forming polymer is an acrylate polymer. Non-limiting examples include acrylates copolymer, styrene/acrylates copolymer, acarylates/ethylhexyl acrylate copolymer, an alkyl acrylate copolymer, acrylic copolymers, polyacrylate-2 crosspolymer, acrylates/hydroxyesters acrylate copolymer, acrylate/ethylhexyl acrylate copolymer, styrene acrylate copolymer, acrylate/VA copolymer, styrene/acrylic copolymer, styrene/acrylates copolymer, styrene/acrylates/ammonium methacrylate copolymer, and a mixture thereof.

Additional non-limiting examples of film forming polymers include but are not limited to: from Kobo Products Inc., DAITOSOL products (e.g., styrene/acrylates copolymer, acarylates/ethylhexyl acrylate copolymer, alkyl acrylate copolymer, etc.), from Dow Chemical Company, EPITEX products (acrylates copolymer), from Interpolymer, SYNTRAN products (acrylic copolymers), from Akzo Nobel Surface Chemistry LLC, Bridgewater N.J., AMPHOMER and AMPHOMER LV-71 polymers (octylacrylamide/acrylates/butylaminoethyl methacrylate compolymer), AMPHOMER HC polymer (acrylates/octylacrylamide copolymer) BALANCE 0/55, BALANCE CR and DERMACRYL AQF polymers (acrylates copolymer), BALANCE 47 polymer (octylacrylamide/butylaminoethyl methacrylate copolymer), RESYN 28-2930 polymer (VA/crotonates/vinyl neodecanoate copolymer), RESYN 28-1310 polymer (VA/Crotonates copolymer), FLEXAN polymers (sodium polystyrene sulfonate), DynamX polymer (polyurethane-14 (and) AMP-Acrylates copolymer), RESYN XP polymer (acrylates/octylacrylamide copolymer), STRUCTURE 2001 (acrylates/steareth-20 itaconate copolymer) and STRUCTURE 3001 (acrylates/ceteth-20 itaconate copolymer); from ISP, OMNIREZ-2000 (PVM/MA half ethyl ester copolymer), GANEX P-904 (butylated PVP), GANEX V-216 (PVP/hexadecene copolymer) GANEX V-220 (PVP/eicosene copolymer), GANEX WP-660 (tricontanyl PVP), GANTREZ A425 (butyl ester of PVM/MA copolymer), GANTREZ AN-119 PVM/MA copolymer, GANTREZ ES 225 (ethyl ester of PVM/MA copolymer), GANTREZ ES425 (butyl ester of PVM/MA copolymer), GAFFIX VC-713 (vinyl caprolactam/PVP/dimethylaminoethyl methacrylate copolymer), GAFQUAT 755 (polyquaternium-11), GAFQUAT HS-100 (polyquaternium-28) AQUAFLEX XL-30 (Polyimide-1), AQUAFLEX SF-40 (PVP/Vinylcaprolactam/DMAPA Acrylates Copolymer), AQUAFLEX FX-64 (Isobutylene/Ethylmaleimide/Hydroxyethylmaleimide Copolymer), ALLIANZ LT-120 (Acrylates/C1-2 Succinates/Hydroxyacrylates Copolymer), STYLEZE CC-10 (PVP/DMAPA Acrylates Copolymer), STYLEZE 2000 (VP/Acrylates/Lauryl Methacrylate Copolymer), STYLEZE W-20 (Polyquaternium-55), Copolymer Series (PVP/Dimethylaminoethylmethacrylate Copolymer), ADVANTAGE S and ADVANTAGE LCA (VinylcaprolactamNP/Dimethylaminoethyl Methacrylate Copolymer), ADVANTAGE PLUS (VA/Butyl Maleate/Isobornyl Acrylate Copolymer); from BASF, ULTRAHOLD STRONG (acrylic acid/ethyl acrylate/t-butyl acrylamide), LUVIMER 100P (t-butyl acrylate/ethyl acrylate/methacrylic acid), LUVIMER 36D (ethyl acrylate/t-butyl acrylate/methacrylic acid), LUVIQUAT HM-552 (polyquaternium-16), LUVIQUAT HOLD (polyquaternium-16), LUVISKOL K30 (PVP) LUVISKOL K90 (PVP), LUVISKOL VA 64 (PVPNA copolymer) LUVISKOL VA73W (PVPNA copolymer), LUVISKOL VA, LUVISET PUR (Polyurethane-1), LUVISET Clear (VP/MethacrylamideNinyl Imidazole Copolymer), LUVIFLEX SOFT (Acrylates Copolymer), ULTRAHOLD 8 (Acrylates/Acrylamide Copolymer), LUVISKOL Plus (Polyvinylcaprolactam), LUVIFLEX Silk (PEG/PPG-25/25 Dimethicone/Acrylates Copolymer); from Amerchol, AMERHOLD DR-25 (acrylic acid/methacrylic acid/acrylates/methacrylates); from Rohm&Haas, ACUDYNE 258 (acrylic acid/methacrylic acid/acrylates/methacrylates/hydroxyl ester acrylates; from Mitsubishi and distributed by Clariant, DIAFORMER Z-301, DIAFORMER Z-SM, and DIAFORMER Z-400 (methacryloyl ethyl betaine/acrylates copolymer), ACUDYNE 180 (Acrylates/Hydroxyesters Acrylates Copolymer), ACUDYNE SCP (Ethylenecarboxyamide/AMPSA/Methacrylates Copolymer), and the ACCULYN rheological modifiers; from ONDEO Nalco, FIXOMER A-30 and FIXOMER N-28 (INCI names: methacrylic acid/sodium acrylamidomethyl propane sulfonate copolymer); from Noveon, FIXATE G-100 (AMP-Acrylates/Allyl Methacrylate Copolymer), FIXATE PLUS (Polyacrylates-X), CARBOPOL Ultrez 10 (Carbomer), CARBOPOL Ultrez 20 (Acrylates/C10-30 Alkyl Acrylates Copolymer), AVALURE AC series (Acrylates Copolymer), AVALURE UR series (Polyurethane-2, Polyurethane-4, PPG-17/IPDI/DMPA Copolymer); polyethylene glycol; water-soluble acrylics; water-soluble polyesters; polyacrylamides; polyamines; polyquaternary amines; styrene maleic anhydride (SMA) resin; polyethylene amine; and other conventional polymer that is polar solvent soluble or that can be made soluble through neutralization with the appropriate base.

The total amount of the secondary, non-polyurethane water-dispersible film forming polymers, if present, may vary but is typically about 1 to about 35 wt. %, based on the total weight of the cosmetic composition. In some instances, the total amount of the secondary film forming polymer may be about 1 to about 30 wt. %, about 1 to about 25 wt. %, about 1 to about 20 wt. %, about 1 to about 15 wt. %, about 1 to about 10 wt. %, about 2 to about 30 wt. %, about 2 to about 25 wt. %, about 2 to about 20 wt. %, about 2 to about 15 wt. %, about 2 to about 10 wt. %, about 3 to about 30 wt. %, about 3 to about 25 wt. %, about 3 to about 20 wt. %, about 3 to about 15 wt. %, or about 3 to about 10 wt. %, based on the total weight of the cosmetic composition.

Pressure Sensitive Adhesive Polymer

Pressure sensitive adhesive polymers provide adhesive properties upon application of pressure. Pressure sensitive adhesive polymers differ somewhat from the film forming polymers discussed above in that they do not typically provide substantial film forming properties. Non-limiting examples of pressure sensitive adhesive polymers include copolymers of acrylate and methacrylate, rubber-based polymers, acrylic polymer, and styrene copolymers (such as styrene-isoprene-styrene (SIS) and styrene-butadiene-styrene (SBS) copolymers). In some instances, the pressure sensitive adhesive polymer is an acrylate polymer, for example, a copolymer of butyl acrylate, butyl methacrylate or acrylic acid. A non-limiting examples of such polymer is 2-ethylhexylacrylate (also referred to as (poly)ethylhexylacrylate) (GEL-TAC 100G, GET-TAC 100F, etc.(Advanced Polymer International)).

Non-limiting examples of pressure sensitive adhesive polymers based on rubber include natural rubber (poly(cis-1,4-isoprene)), methyl methacrylate-isoprene graft copolymers, styrene-butadiene copolymer, butyl rubber, acrylonitrile-butadiene rubber, styrene-isoprene block copolymer, polybutadiene, ethylene-butylene block copolymer, and polychloroprene.

Another type of pressure sensitive adhesive polymer that may be used includes those based on polar acrylic polymers. For example, these can include statistical or block copolymers based on acrylic acid, alkyl acrylates, and alkyl methacrylates can be used, as well as copolymers of these acrylics with ethylene and vinyl acetate.

Specifically preferred are copolymers of butyl acrylate, butyl methacrylate, and acrylic acid. These copolymers are commercially available, for example, under the name Roderm 560, including Acudyne 5800P (a modified version of Roderm 580)(Dow). Another preferred pressure sensitive adhesive according to the invention comprises poly(2-ethylhexylacrylate), which is, for example, commercially available under the name Gel-Tac 100G (Advanced Polymer International) as a 40% solids aqueous dispersion of 15 micron tacky acrylic microspheres or Gel-Tac 100F (Advanced Polymers International) as a 45% solids aqueous dispersion of 29 micron tacky acrylic microspheres

Specific examples of useful acrylic copolymers are commercially available under the trade names EASTAREZ 2010, 2020, and 2050 (Eastman Chemical Co.); ACRONAL V210 (BASF); Mowilith LDM 7255 and Revacryl 491 (Clariant); and FLEXBOND 165 (Air Products). Specific examples of useful rubber polymeric materials (isoprene and butadiene polymers) are commercially available under the trade names RICON 130 polybutadiene (Atofina Sartomer) and ISOLENE 40 polyisoprene (Elementis). Specific examples of useful vinyl acetate copolymers are commercially available under the trade names PVP/VA S-630 (International Specialty Products) and FLEXBOND 149 (Air Products). Specific examples of useful vinyl alcohol/vinyl acetate copolymers are commercially available under the trade names CELVOL 107 (CELANESE) and ELVANOL 50-42 (DuPont).

The total amount of the pressure sensitive adhesive polymer may vary but is typically about 1 to about 35 wt. %, based on the total weight of the cosmetic composition. In some instance, the total amount of the pressure sensitive adhesive polymer may be about 1 to about 30 wt. %, about 1 to about 25 wt. %, about 1 to about 20 wt. %, about 1 to about 15 wt. %, about 1 to about 10 wt. %, about 2 to about 30 wt. %, about 2 to about 25 wt. %, about 2 to about 20 wt. %, about 2 to about 15 wt. %, about 2 to about 10 wt. %, about 3 to about 30 wt. %, about 3 to about 25 wt. %, about 3 to about 20 wt. %, about 3 to about 15 wt. %, or about 3 to about 10 wt. %, based on the total weight of the cosmetic composition.

Soft Focus Powder

Soft focus powders are materials providing a blurring effect, typically due to their light-scattering properties on the skin. Such powders typically have high diffuse reflectance, low specular reflectance, and high diffuse transmittance. Soft focus powders give the skin a smoother appearance, for example, by reducing the difference in luminosity between the valley and the edges of wrinkles and imperfections.

Non-limiting examples of soft focus powders include powders of natural or synthetic origin such as mica, titanated mica, alumina, aluminum silicate, silica which may or may not be coated, fumed silica, silica silylate, titanium dioxide, serecite, composite talc/titanium dioxide/alumina/silica powders, polyamide, poly(methyl (meth)acrylate), polyethylene powder, polymethylsilsesquioxane powder, and a mixture thereof. Additional non-limiting examples include calcium aluminum borosilicate (LUXSIL), PMMA (Microsphere M-100), polyethylene (POLYETHYLENE CI 2080), methyl methacrylate crosspolymer (COVABEADS LH85), nylon-12 (ORGASOL 2002), or ethylene/acrylic acid copolymer (FLOBEADS EA209). In some instances, the cosmetic compositions include at least one soft focus powder selected from the group consisting of silica which may or may not be coated, fumed silica, silica silylate, composite talc/titanium dioxide/alumina/silica powders, polyamide (nylon), poly(methyl (meth)acrylate), polyethylene powder, polymethylsilsesquioxane powder, waxes, such as copernicia cerifera (carnauba) wax, dimethicone/vinyl dimethicone crosspolymer, nylon-12, cellulose, polylactic acid, boron nitride, and a mixture thereof. The copernicia cerifera (carnauba) wax can be provided as a dispersion non water and alcohol. The dimethicone/vinyl dimethicone crosspolymer can be provided as silicone dispersion (INCI: Dimethicone/vinyl dimethicone crosspolymer (and) C12-14 Pareth-12).

The total amount of soft focus powder can vary but is typically about 1 to about 35 wt. %, based on the total weight of the cosmetic composition. In some cases, the total amount of soft focus powder is about 1 to about 30 wt. %, about 1 to about 25 wt. %, about 1 to about 20 wt. %, about 5 to about 35 wt. %, about 5 to about 30 wt. %, about 5 to about 25 wt. %, about 5 to about 20 wt. %, about 10 to about 35 wt. %, about 10 to about 30 wt. %, about 10 to about 25 wt. %, or about 10 to about 20 wt. %, based on the total weight of cosmetic composition.

Thickening Agents

At least one thickening agent (also referred to as thickeners or viscosity modifying agents) can be included in the compositions. In some instances, the compositions include at least one hydrophilic thickening agent. Non-limiting examples include modified or unmodified carboxyvinyl polymers (e.g., carbomer), acrylates/C10-30 alkyl acrylate crosspolymer, polyacrylates, polymethacrylates, polyacrylamides, optionally crosslinked and/or neutralized 2-acrylamido-2-methylpropane sulfonic acid polymers (e.g., ammonium polyacryldimethyltauramide), crosslinked anionic copolymers of acrylamide and of 2-acrylamido-2-methylpropane sulfonic acid (AMPS) (e.g., acrylamide/sodium acryloyldimethyltaurate copolymer), polysaccharides (e.g., xanthan gum, guar gum, carob gum, acacia gum, scleroglucans, chitin and chitosan derivatives, carranenans, gellans, alginates), celluloses (e.g., microcrystalline cellulose, carboxymethylcellulose, hydroxymethylcellulose and hydroxypropylcellulose), and a mixture thereof. In some instances, a preferred thickening agent is a crosslinked anionic copolymer of acrylamide and of 2-acrylamido-2-methylpropane sulfonic acid (AMPS) (e.g., acrylamide/sodium acryloyldimethyltaurate copolymer).

Additional non-limiting examples of hydrophilic thickening agents include polyvinylpyrrolidone (PVP), polyvinyl alcohol, crosslinked acrylates (e.g. CARBOPOL 982), hydrophobically-modified acrylates (e.g. Carbopol 1382); polyacrylamides such as, for example, the crosslinked copolymers sold under the names Sepigel 305 (CTFA name: polyacrylamide/C13-C14 isoparaffin/Laureth 7) or SIMULGEL 600 (CTFA name: acrylamide/sodium acryloyldimethyltaurate copolymer/isohexadecane/polysorbate 80) by SEPPIC; 2-acrylamido-2-methylpropanesulphonic acid polymers and copolymers, that are optionally crosslinked and/or neutralized; cellulose derivatives such as hydroxyethylcellulose, sodium carboxymethylcellulose, hydroxypropyl methylcellulose, hydroxypropyl cellulose, ethyl cellulose and hydroxymethyl cellulose; polysaccharides and gums, e.g., natural gums such as xanthan gum, sclerotium, carrageenan and pectin; polysaccharide resins such as starch and its derivatives, hyaluronic acid and its salts, clays, and, in particular, montmorillonites, hectorites, bentonites, and laponites, crosslinked polyacrylic acids, such as the “CARBOPOL” products from the company Goodrich, the polyglyceryl(meth)acrylates polymers sold under the names “HISPAGEL” or “LUBRAGEL” by the companies Hispano Quimica or Guardian, crosslinked acrylamide polymers and copolymers, such as those sold under the names “PAS 5161” or “BOZEPOL C” by the company Hoechst, “SEPIGEL 305” by the company SEPPIC, crosslinked methacryloyloxyethyltrimethylammonium chloride homopolymers sold under the name “SALCARE SC95” by the company Allied Colloid, and a mixture thereof.

A more exhaustive list of useful thickening agents that may be included in the cosmetic compositions is provided later, under the heading “Additional Thickening Agents.”

Water-Soluble Solvents

The aqueous phase of the cosmetic composition typically includes one or more water-soluble solvents. The term “water-soluble solvent” is interchangeable with the term “water-miscible solvent” and means a compound that is liquid at 25° C. and at atmospheric pressure (760 mmHg), and it has a solubility of at least 50% in water under these conditions. In some cases, the water soluble solvent has a solubility of at least 60%, 70%, 80%, or 90%. Non-limiting examples of water-soluble solvents include, for example, glycerin, C₁₋₄ alcohols, organic solvents, fatty alcohols, fatty ethers, fatty esters, polyols, glycols, and any a mixture thereof. In some instances, the cosmetic composition includes one or more C₁₋₄ alcohols, for example, ethanol.

As examples of organic solvents, non-limiting mentions can be made of monoalcohols and polyols such as ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, propane diol, and glycerin. The organic solvents can be volatile or non-volatile compounds.

Further non-limiting examples of water-soluble solvents include alkanediols (polyhydric alcohols) such as glycerin, 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, and a mixture thereof.

Polyhydric alcohols are useful. Examples of polyhydric alcohols include glycerin, ethylene glycol, diethylene glycol, triethylene glycol, propylene glycol, dipropylene glycol, tripropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, and a mixture thereof. Polyol compounds may also be used. Non-limiting examples include the aliphatic diols, such as 2-ethyl-2-methyl-1,3-propanediol, 3,3-dimethyl-1,2-butanediol, 2,2-diethyl-1,3-propanediol, 2-methyl-2-propyl-1,3-propanediol, 2,4-dimethyl-2,4-pentanediol, 2,5-dimethyl-2,5-hexanediol, 5-hexene-1,2-diol, and 2-ethyl-1,3-hexanediol, and a mixture thereof.

The total amount of the water-soluble solvents in the cosmetic composition may vary but is typically about 0.01 to about 25 wt. %, based on the total weight of the cosmetic composition. In some cases, the total amount of water-soluble solvents is about 0.01 to about 20 wt. %, about 0.01 to about 15 wt. %, about 0.01 to about 10 wt. %, about 0.1 to about 25 wt. %, about 0.1 to about 20 wt. %, about 0.1 to about 15 wt. %, about 0.1 to about 10 wt. %, about 1 to about 25 wt. %, about 1 to about 20 wt. %, about 1 to about 15 wt. %, about 1 to about 10 wt. %, or about 1 to about 8 wt. %, based on the total weight of the cosmetic composition.

The total amount of water in the cosmetic compositions may vary but is typically about 10 to about 90 wt. %, based on the total weight of the cosmetic composition. Additionally, the total amount of water may be about 20 to about 80 wt. %, about 25 to about 75 wt. %, about 30 to about 70 wt. %, about 35 to about 65 wt. %, or about 40 to about 60 wt. %, based on the total weight of the cosmetic composition.

The cosmetic compositions may include copolymers of polydimethylsiloxane, for example, high molecular weight copolymers of polydimethylsiloxane. Non-limiting examples include divinyldimethicone/dimethicone copolymers, such as HMW 2200 from Dow Corning (divinyldimethicone/dimethicone copolymer (and) C12-12 Pareth-3 (and) C12-13 Pareth-2.

The cosmetic compositions may include one or more surfactants, for example, one or more nonionic, anionic, cationic, amphoteric (zwitterionic) surfactants. Likewise, the composition may exclude one or more surfactants or types of surfactants. For example, the cosmetic compositions may be free or essentially free of all types of surfactants or may be free or essentially free of nonionic surfactant, anionic surfactants, cationic surfactants, or amphoteric surfactants.

The cosmetic compositions may include one or more oils or waxes. Nonetheless, the cosmetic compositions may also be free or essentially free of oils and/or waxes.

In one embodiment, cosmetic composition according to the instant disclosure may include:
about 1 to about 40 wt. %, about 5 to about 40 wt. %, or about 5 to about 30 wt. % of at least one primary water-dispersible film forming polymer, preferably at least two primary water-dispersible film forming polymers, having a Young's modulus of 0.1 to 50 MPa, an elongation at break >50% and a glass transition temperature of <0° C., for example at least one polyurethane latex polymer such as polyurethane-34 and/or polyurethane-35;
optionally, about 1 to about 35 wt. %, about 1 to about 25 wt. %, or about 1 to about 15 wt. % of at least one secondary, non-polyurethane water-dispersible film forming polymer, for example, an acrylate polymer, having a glass transition temperature of <25° C.,
   wherein the glass transition of the at least one primary water-dispersible film forming polymer and the at least one secondary film forming polymer, if present, differs by at least 20° C.;
about 1 to about 35 wt. %, about 1 to about 25 wt. %, or about 1 to about 15 wt. % of at least one pressure sensitive adhesive polymer, for example, a pressure sensitive adhesive acrylate polymer that is a copolymer of butyl acrylate, butyl methacrylate or acrylic acid (e.g., 2-ethylhexylacrylate);
about 1 to about 35 wt. %, about 1 to about 20 wt. %, or about 5 to about 20 wt. % of soft focus powder, for example, talc, mica, titanated mica, alumina, aluminum silicate, silica which may or may not be coated, fumed silica, silica silylate, polyamide, poly(methyl (meth)acrylate), polyethylene powder, polymethylsilsesquioxane powder, and a mixture thereof;
about 0.01 to about 10 wt. %, about 0.01 to about 5 wt. %, or about 0.1 to about 5 wt. % of at least one thickening agent, for example acrylamide/sodium acryloyldimethyltaurate copolymer;
optionally, about 0.01 to about 25 wt. %, about 0.1 to about 20, or about 1 to about 10 wt. % of at least one water soluble solvent, for example, glycerin, a C1-4 alcohol, an organic solvent, a polyol, a glycol, and a mixture thereof; and
25 to about 75 wt. %, about 30 to about 70 wt. %, or about 35 to about 75 wt. % of water;
   wherein the weight percentages are based on the total weight of the cosmetic composition.

The cosmetic composition of the above embodiment may include a total amount of film forming polymers (primary water-dispersible film forming polymers and secondary water-dispersible film forming polymer(s)) of at least 10 wt. %, based on the total weight of the cosmetic composition. The total amount of film forming polymers may be at least 15 wt. %, at least 20 wt. %, or at least 25 wt. %, based on the total weight of the cosmetic composition. The maximum amount of total film forming polymers is typically not greater than about 30, 40, or 50 wt. %, based on the total weight of the cosmetic composition.

The weight ratio of total amount of all primary water-dispersible film forming polymers to the total amount of all secondary film forming polymers is at least 2:1. For example, the total amount of all primary water-dispersible film forming polymer to the total amount of all secondary film forming polymers may be at least 2:1 to about 10:1, at least 2:1 to about 8:1, at least 2:1 to about 6:1, about 3:1 to about 10:1, about 3:1 to about 8:1, about 3:1 to about 7:1, or about 4:1 to about 7:1.

In one embodiment, the cosmetic composition of the instant disclosure may include:
about 1 to about 25 wt. %, about 2 to about 20 wt. %, or about 5 to about 20 wt. % of a first primary water-dispersible film forming polymer having a Young's modulus of 0.1 to 10 MPa, and elongation at break >50% and a glass transition temperature of <0° C., for example, polyurethane-34;
about 5 to about 20 wt. % of a second primary water-dispersible film forming polymer having a Young's modulus of greater than 10 to 50 MPa, and elongation at break >50% and a glass transition temperature of <0° C., for example, polyurethane-35;
optionally, about 1 to about 25 wt. %, about 1 to about 20, or about 1 to about 15 wt. % of a secondary, non-polyurethane film forming polymer having a glass transition temperature of <25° C. that is an acrylate polymer, for example, an acrylate polymer selected from the group consisting of acrylates copolymer, styrene/acrylates copolymer, acarylates/ethylhexyl acrylate copolymer, an alkyl acrylate copolymer, acrylic copolymers, styrene/acrylates copolymer, acarylates/ethylhexyl acrylate copolymer, an alkyl acrylate copolymer, acrylic copolymers, polyacrylate-2 crosspolymer, acrylates/hydroxyesters acrylate copolymer, acrylate/ethylhexyl acrylate copolymer, styrene acrylate copolymer, acrylate/VA copolymer, styrene/acrylic copolymer, styrene/acrylates copolymer, styrene/acrylates/ammonium methacrylate copolymer, and a mixture thereof;
   wherein the glass transition of the at least one primary water-dispersible film forming polymer and the at least one secondary film forming water-dispersible polymer, if present, differs by at least 20° C.;
about 1 to about 25 wt. %, about 1 to about 20, or about 1 to about 15 wt. % of a pressure sensitive adhesive polymer that is a copolymer of butyl acrylate, butyl methacrylate or acrylic acid, for example, 2-ethylhexylacrylate;
about 1 to about 35 wt. %, about 5 to 30 wt. %, or about 5 to about 30 wt. % of soft focus powder, for example, a soft focus powder selected from the group consisting of talc, mica, titanated mica, alumina, aluminum silicate, silica which may or may not be coated, fumed silica, silica silylate, polyamide, poly(methyl (meth)acrylate), polyethylene powder, polymethylsilsesquioxane powder, and a mixture thereof, wherein silica and/or polymethylsilsesquioxane powder are preferred;
about 0.01 to about 5 wt. %, about 0.1 to about 5 wt. %, or about 0.1 to about 3 wt. % of at least one hydrophilic thickening agent, for example, a hydrophilic thickening agent selected from the group consisting of modified or unmodified carboxyvinyl polymers (e.g., carbomer), acrylates/C10-30 alkyl acrylate crosspolymer, polyacrylates, polymethacrylates, polyacrylamides, optionally crosslinked and/or neutralized 2-acrylamido-2-methylpropane sulfonic acid polymers (e.g., ammonium polyacryldimethyltauramide), crosslinked anionic copolymers of acrylamide and of 2-acrylamido-2-methylpropane sulfonic acid (AMPS) (e.g., acrylamide/sodium acryloyldimethyltaurate copolymer), polysaccharides (e.g., xanthan gum, guar gum, carob gum, acacia gum, scleroglucans, chitin and chitosan derivatives, carranenans, gellans, alginates), celluloses (e.g., microcrystalline cellulose, carboxymethylcellulose, hydroxymethylcellulose and hydroxypropylcellulose), and a mixture thereof;
about 0.01 to about 25 wt. % of at least one water soluble solvent, for example, glycerin, a C1-4 alcohol, an organic solvent, a polyol, a glycol, and a mixture thereof; and
25 to about 75 wt. %, about 30 to about 70 wt. %, or about 35 to about 75 wt. % of water;
   wherein the weight percentages are based on the total weight of the cosmetic composition.

The cosmetic composition of the above embodiment may include a total amount of film forming polymers (primary and secondary water-dispersible film forming polymer(s)) of at least 10 wt. %, based on the total weight of the cosmetic composition. The total amount of film forming polymers may be at least 15 wt. %, at least 20 wt. %, or at least 25 wt. %, based on the total weight of the cosmetic composition. The maximum amount of total film forming polymers is typically not greater than about 30, 40, or 50 wt. %, based on the total weight of the cosmetic composition.

The weight ratio of total amount of all primary water-dispersible film forming polymers (polyurethane-34 and polyurethane-35) to the total amount of all secondary water-dispersible film forming polymers is at least 2:1. For example, the total amount of all primary film forming polymer to the total amount of all secondary film forming polymers may be at least 2:1 to about 10:1, at least 2:1 to about 8:1, at least 2:1 to about 6:1, about 3:1 to about 10:1, about 3:1 to about 8:1, about 3:1 to about 7:1, or about 4:1 to about 7:1.

Additional Thickening Agents

The one or more thickening agents may be xanthan gum, guar gum, biosaccharide gum, cellulose, acacia Seneca gum, sclerotium gum, agarose, pechtin, gellan gum, hyaluronic acid. Additionally, the one or more thickeners may include polymeric thickeners selected from the group consisting of ammonium polyacryloyldimethyl taurate, ammonium acryloyldimethyltaurate/VP copolymer, sodium polyacrylate, acrylates copolymers, polyacrylamide, carbomer, and acrylates/C10-30 alkyl acrylate crosspolymer. In some cases, the thickening agent includes ammonium polyacryloyldimethyl taurate and/or sodium polyacrylate.

Many thickening agents are water-soluble, and increase the viscosity of water or form an aqueous gel when the cosmetic composition of the invention is dispersed/dissolved in water. The aqueous solution may be heated and cooled, or neutralized, for forming the gel, if necessary. The thickener may be dispersed/dissolved in an aqueous solvent that is soluble in water, e.g., ethyl alcohol when it is dispersed/dissolved in water. Non-limiting examples of various types of thickeners include:

a. Carboxylic Acid Polymers

These polymers are crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol.

Examples of commercially available carboxylic acid polymers useful herein include the carbomers, which are homopolymers of acrylic acid crosslinked with allyl ethers of sucrose or pentaerytritol. The carbomers are available as the Carbopol® 900 series from B.F. Goodrich (e.g., Carbopol® 954). In addition, other suitable carboxylic acid polymeric agents include Ultrez® 10 (B.F. Goodrich) and copolymers of C10-30 alkyl acrylates with one or more monomers of acrylic acid, methacrylic acid, or one of their short chain (i.e., C1-4 alcohol) esters, wherein the crosslinking agent is an allyl ether of sucrose or pentaerytritol. These copolymers are known as acrylates/C10-C30 alkyl acrylate crosspolymers and are commercially available as Carbopol® 1342, Carbopol® 1382, Pemulen TR-1, and Pemulen TR-2, from B.F. Goodrich. In other words, examples of carboxylic acid polymer thickeners useful herein are those selected from carbomers, acrylates/C10-C30 alkyl acrylate crosspolymers, and mixtures thereof.

b. Crosslinked Polyacrylate Polymers

The compositions of the present disclosure can optionally contain crosslinked polyacrylate polymers useful as thickeners or gelling agents including both cationic and nonionic polymers. Examples of useful crosslinked nonionic polyacrylate polymers and crosslinked cationic polyacrylate polymers are those described in U.S. Pat. Nos. 5,100,660, 4,849,484, 4,835,206, 4,628,078 4,599,379 and EP 228,868, which are all incorporated herein by reference in their entirety.

c. Polyacrylamide Polymers

The compositions of the present disclosure can optionally contain polyacrylamide polymers, especially nonionic polyacrylamide polymers including substituted branched or unbranched polymers. Among these polyacrylamide polymers is the nonionic polymer given the CTFA designation polyacrylamide and isoparaffin and laureth-7, available under the Tradename Sepigel 305 from Seppic Corporation.

Other polyacrylamide polymers useful herein include multi-block copolymers of acrylamides and substituted acrylamides with acrylic acids and substituted acrylic acids. Commercially available examples of these multi-block copolymers include Hypan SR150H, SS500V, SS500W, SSSA100H, from Lipo Chemicals, Inc.

The compositions may also contain thickening and texturising gels of the type as exemplified by the product range called Lubrajel® from United Guardian. These gels have moisturizing, viscosifying, stabilizing properties.

d. Polysaccharides

A wide variety of polysaccharides can be useful herein. “Polysaccharides” refer to gelling agents that contain a backbone of repeating sugar (i.e., carbohydrate) units. Nonlimiting examples of polysaccharide gelling agents include those selected from the group consisting of cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Also useful herein are the alkyl-substituted celluloses. Preferred among the alkyl hydroxyalkyl cellulose ethers is the material given the CTFA designation cetyl hydroxyethylcellulose, which is the ether of cetyl alcohol and hydroxyethylcellulose. This material is sold under the tradename Natrosol® CS Plus from Aqualon Corporation.

Other useful polysaccharides include scleroglucans comprising a linear chain of (1-3) linked glucose units with a (1-6) linked glucose every three units, a commercially available example of which is Clearogel™. CS11 from Michel Mercier Products Inc.

e. Gums

Other thickening and gelling agents useful herein include materials which are primarily derived from natural sources. Nonlimiting examples of these gelling agent gums include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, and mixtures thereof.

Additional examples of water-soluble thickeners include water-soluble natural polymers, water-soluble synthetic polymers, clay minerals and silicic anhydride. Non-limiting examples of water-soluble natural polymers include gum arabic, tragacanth gum, karaya gum, guar gum, gellan gum, tara gum, locust bean gum, tamarind gum, sodium alginate, alginic acid propyleneglycol ester, carrageenan, farcelluran, agar, high-methoxy pectin, low-methoxy pectin, xanthine, chitosan, starch (for example starch derived from corn, potato, wheat, rice, sweet potato and tapioca, a-starch, soluble starch), fermentation polysaccharide (for example, xanthan gum, pullulan, carciran, dextran), acidic hetero-polysaccharide derived from callus of plants belonging to Polyantes sp. (for example, tuberous polysaccharide), proteins (for example, sodium casein, gelatin, albumin), chondroitin sulfate, and hyaluronic acid.

Non-limiting examples of water-soluble synthetic polymers include polyvinyl alcohol, sodium polyacrylate, sodium polymethacrylate, polyacrylic acid glycerin ester, carboxyvinyl polymer, polyacrylamide, polyvinyl pyrrolidone, polyvinyl methylether, polyvinyl sulfone, maleic acid copolymer, polyethylene oxide, polydiallyl amine, polyethylene imine, water soluble cellulose derivatives (for example, carboxymethyl cellulose, methyl cellulose, methylhydroxypropyl cellulose, hydroxyethyl cellulose, hydroxypropyl cellulose, cellulose sulfate sodium salt), and starch derivatives (for example, starch oxide, dialdehyde starch, dextrin, British gum, acetyl starch, starch phosphate, carboxymethyl starch, hydroxyethyl starch, hydroxypropyl starch).

In some instances, additional ingredients are also present in the cosmetic compositions. Non-limiting examples of additional ingredients that may be optionally present include preservatives (e.g., phenoxyethanol, ethyl hexylglycerin, etc.), coalescents, wetting agents (e.g., PEG 12 dimethicone), emollients, etc.

The compositions described herein may be in the form of a crème, a gel, a wax, a lotion, etc. In some instances, the composition is not a mousse. Furthermore, the compositions may be free or essentially free of propellants

Implementation of the present disclosure is provided by way of the following examples. The examples serve to illustrate the technology without being limiting in nature.

### EXAMPLE 1

(Skin Perfecting Composition)

| | | A |
|---|---|---|
| | INCI US | WT. % |
| Primary Water- | POLYURETHANE-34 | 11.4 |
| Dispersible Film | | |
| Forming Polymer | | |
| Primary Water- | POLYURETHANE-35 | 11.4 |
| Dispersible Film | | |
| Forming Polymer | | |
| Secondary Film | ACRYLATES COPOLYMER | 4.5 |
| Forming Polymer | | |
| Pressure Sensitive | 2-ETHYLHEXYL ACRYLATE | 4.5 |
| Adhesive (PSA) | (Polymer of the monomer 2- | |
| Polymer | ethylhexyl acrylate) | |
| Soft focus powders | POLYMETHYLSILSESQUIOXANE | 13 |
| | AND/OR SILICA | |
| Thickener | ACRYLAMIDE/SODIUM | 0.4 |
| | ACRYLOYLDIMETHYLTAURATE | |
| | COPOLYMER | |
| Solvent | ISOHEXADECANE | 0.2 |
| Water-Soluble | ALCOHOL DENAT. | 3 |
| Solvent | | |
| Nonionic Surfactant | LAURETH-21, POLYSORBATE | ≤1 |
| | 80, & SORBITAN OLEATE | |
| Preservatives | PRESERVATIVES | ≤1 |
| Water | WATER | QS 100 |

### EXAMPLE 2

(Expert Evaluations)

| | | B | C | D | E | F | G |
|---|---|---|---|---|---|---|---|
| | | wt. % | wt. % | wt. % | wt. % | wt. % | wt. % |
| | INCI US | 1 | 2 | 3 | 4 | 5 | 6 |
| Primary Film | POLYURETHANE-34 | 13 | 11.4 | 11.4 | 11.4 | 5 | 12.9 |
| Forming Polymer | Tg = −51.5° C. | | | | | | |
| (Bayusan C 1001) | | | | | | | |
| Primary Film | POLYURETHANE-35 | 13 | 11.4 | 11.4 | 11.4 | 5 | 12.9 |
| Forming Polymer | Tg = −45.5° C. | | | | | | |
| (Bayusan C 1004) | | | | | | | |
| Secondary Film | ACRYLATES | — | 4.5 | 4.5 | 4.5 | — | — |
| Forming Polymer | COPOLYMER | | | | | | |
| Daitosol 5000AD | Tg = −10.2° C. | | | | | | |
| Soft Focus | POLYMETHYLSILSES | 9 | 9 | 9 | 9 | 3.5 | 9 |
| Particles | QUIOXANE | | | | | | |
| | SILICA | 4 | 4 | 4 | 4 | 1.5 | 4 |
| PSA Polymer | 2-ETHYLHEXYL | 4.5 | 4.5 | 4.5 | 4.5 | 1.7 | 4.5 |
| (Gel-Tac100 | ACRYLATE | | | | | | |
| Acrylate | Tg = −71.2° C. | | | | | | |
| Microspheres) | | | | | | | |
| Thickener | ACRYLAMIDE/SODIUM | 0.4 | 0.4 | 0.4 | 0.4 | 0.7 | 0.4 |
| | ACRYLOYLDIMETHYL | | | | | | |
| | TAURATE | | | | | | |
| | COPOLYMER | | | | | | |
| Solvent | ISOHEXADECANE | 0.2 | 0.2 | 0.2 | 0.2 | 0.4 | 0.2 |
| Water-Soluble | ALCOHOL DENAT. | 3 | 3 | 3 | 3 | 3 | 3 |
| Solvent | | | | | | | |
| Nonionic Surfactant | LAURETH-21, | 0.1 | 0.1 | 0.1 | 0.1 | 0.2 | 0.1 |
| | POLYSORBATE 80, & | | | | | | |
| | SORBITAN OLEATE | | | | | | |
| Coalescent | DIPROPYLENE | — | — | 1 | — | — | — |
| | GLYCOL DIBENZOATE | | | | | | |
| Wetting agent | PEG-12 DIMETHICONE | — | — | — | 0.5 | — | 0.5 |
| Preservatives | Preservatives | ≤1 | ≤1 | ≤1 | ≤1 | ≤1 | ≤1 |
| Water | WATER | QS | QS | QS | QS | QS | QS |
| | | 100 | 100 | 100 | 100 | 100 | 100 |

Composition G was tested on a panel of 5 individuals having grade 4 eye bags (based on Atlas' scores of 0 to 5). The individuals' faces were cleansed and the skin allowed to completely dry and acclimate for 10 minutes. After 10 minutes, a standard facial moisturizing composition was applied to the skin. The moisturizing composition was applied in order to mimic a regular skin care routine. Approximately 2 minutes after application of the moisturizing composition, Composition G was applied to the area under the eyes, the glabella lines and to the forehead.

A panel of cosmetologists evaluated the skin at baseline, 10 min., 30 min., 3 hours, and 6 hours. Each of the applicable attributes were graded based on a skin aging Atlas (Roland Bazin, Eric Doublet, Skin Aging ATLAS, Vol. 1 (Caucasian Type), MED'COM (2007)) on a scale of 0 to 5 (none to severe). The results are summarized in the table below. The value in parenthesis shows the reduction from baseline.

| | Baseline | 10 min | 3 hrs | 6 hrs |
|---|---|---|---|---|
| Eye bags | 4 | 1.6 | 1.2 | 1.4 |
| | | (−2.4) | (−2.8) | (−2.6) |
| Under eye | 3.6 | 0.8 | 0.8 | 0.8 |
| wrinkles | | (−2.8) | (−2.8) | (−2.8) |
| Crow's feet | 3.0 | 0 | 0.8 | 0.8 |
| | | (−3.0) | (−2.2) | (−1.8) |
| Glabellar lines | 3.0 | 0.8 | 0.8 | 1.0 |
| | | (−2.2) | (−2.2) | (−2.0) |
| Forehead | 3.0 | 1.0 | 1.0 | 1.2 |
| | | (−2.0) | (−2.0) | (−1.8) |
| Shine | 0 | 0 | 0 | 0 |

The results show that application of Composition G significantly improved (reduced the appearance of) eye bags, under eye wrinkles, crow's feet, glabellar lines, and forehead lines. The improvements lasted for at least 6 hours. Negative numbers represent a reduction from baseline.

### EXAMPLE 3

(Functional Properties)

| | | B | H | I |
|---|---|---|---|---|
| | INCI US | wt. % | wt. % | wt. % |
| Primary Film Forming | POLYURETHANE-34 | 13 | 13 | 13 |
| Polymer | Tg = −51.5° C. | | | |
| (Bayusan C 1001) | | | | |
| Primary Film Forming | POLYURETHANE-35 | 13 | 13 | 13 |
| Polymer | Tg = −45.5° C. | | | |
| (Bayusan C 1004) | | | | |
| Soft Focus Particles | POLYMETHYL- | 9 | 9 | 9 |
| | SILSESQUIOXANE | | | |
| Soft Focus Particles | SILICA | 4 | 4 | 4 |
| Pressure Sensitive | 2-ETHYLHEXYL | 4.5 | | |
| Adhesive (PSA) | ACRYLATE | | | |
| Polymer | Tg = −71.2° C. | | | |
| (Gel-Tac100 Acrylate | | | | |
| Microspheres) | | | | |
| Pressure Sensitive | ACRYLIC POLYMER | | 4.5 | |
| Adhesive (PSA) | Tg = −41 deg C. | | | |
| (Acudyne 5800P | | | | |
| Acrylic Polymer | | | | |
| Microspheres) | | | | |
| Thickener | ACRYLAMIDE/SODIUM | 0.4 | 0.4 | 0.4 |
| | ACRYLOYLDIMETHYL- | | | |
| | TAURATE COPOLYMER | | | |
| Solvent | ISOHEXADECANE | 0.2 | 0.2 | 0.2 |
| Water-Soluble Solvent | ALCOHOL DENAT. | 3 | 3 | 3 |
| Nonionic Surfactant | POLYSORBATE 80, & | 0.1 | 0.1 | 0.1 |
| | SORBITAN OLEATE | | | |
| Preservatives | PRESERVATIVES | ≤1 | ≤1 | ≤1 |
| Water | WATER | QS | QS | QS |
| | | 100 | 100 | 100 |

The compositions were tested for three characteristics: (1) affinity with sebum (contact angle); (2) shine when exposed to sebum (gloss); and (3) lastingness of skin.

To determine the affinity with sebum (contact angle), the compositions were applied to 50 μm polyester sheets (BYK # 2871) with an 8 mil drawn down bar and left to dry for 1 hour. Contact angle measurements were taken at 1 hour dry time using a Attension Optical Tensiometer (Biolin Scientific). A 3 μL droplet of artificial sebum was placed on the dried surface of the films formed by the compositions, and the Young-Laplace contact angle was measured after 10 sec using OneAttension software (V 2.3, Biolin Scientific). Contact angle values are reported as the average of 3 samples (N=3).

To measure shine (gloss), the compositions were applied to black contrast cards with an 8 mil draw down bar and allowed to dry for 1 hour. Once dried, a drop of artificial sebum was spread onto the surface of the films covering an area of about 4 by 6 cm and the degree of gloss visually determined.

To determine the lastingness (durability) of the films formed by the compositions on skin, each composition was to the forehead and under the eyes of volunteers and allowed to dry for 1 hour. After 1 hour, 0.5 ml of an oil-free makeup remover was dispensed on half a disk of cotton pad and the number of swipes required to totally remove the composition with the cotton pad were counted. The values reported are the average number of swipes for 2 trials. The results are presented in the table below.

| | B | H | I |
|---|---|---|---|
| Affinity with Sebum | 16.6° | 23.7° | <10° |
| (contact angle) | | | |
| Shine when exposed | Low Shine | Very Shiny | Very Shiny |
| to sebum (in vitro) | | | |
| Lastingness on skin | 19 swipes on | 24.5 swipes on | 18 swipes on |
| | forehead; | forehead; | forehead; |
| | 15 swipe under eye; | 20 swipes under eye; | 11 swipes under eye; |
| | Several sticky | Several sticky | Removal in one |
| | pieces | | piece |

The affinity with sebum is quantified by contact angle. A lower contact angle represents a higher affinity with sebum. A higher affinity with sebum is less desirable because it results in less sebum resistance. Therefore, a higher contact angle represent a better sebum resistance. Compositions B and H provided better sebum resistance than Composition I. With respect to shine, Composition B provided less shininess than Compositions H and I. Lastingness on skin was determined using cotton swabs that were wetted with a commercial make-up removing compositions. It is desirable for the compositions to exhibit lastingness of the skin and not easily rub off. Compositions B and H required more swipes for removes, i.e., Compositions B and H were more difficult to remove from the skin than Composition I. The results indicate that inclusion of a pressure sensitive adhesive improves sebum resistance, reduces shine, and improves lastingness.

### EXAMPLE 4

(Comparative Testing)

| Skin Perfecting Composition | | | | |
|---|---|---|---|---|
| | | J | C-1 | C-2 |
| | INCI US | wt. % | wt. % | wt. % |
| Primary Film | POLYURETHANE-34 | 10 | | 10 |
| Forming Polymer | Tg = −51.5° C. | | | |
| (Bayusan C 1001) | | | | |
| Primary Film | POLYURETHANE-35 | 10 | | |
| Forming Polymer | Tg = −45.5° C. | | | |
| (Bayusan C 1004) | | | | |
| Secondary Film | ACRYLATES COPOLYMER | | | 10 |
| Forming Polymer | Tg = −10.2° C. | | | |
| (Daitosol 5000AD) | | | | |
| Primary Film | POLYURETHANE-2 (and) | | 20 | |
| Forming Polymer | POLYMETHYL | | | |
| (Hybridur) | METHACRYLATE | | | |
| Soft Focus | POLYMETHYL- | 9 | 9 | 9 |
| Particles | SILSESQUIOXANE | | | |
| | SILICA | 4 | 4 | 4 |
| PSA Polymer (Gel- | 2-ETHYLHEXYL | 4.5 | 4.5 | 4.5 |
| Tac100 Acrylate | ACRYLATE | | | |
| Microspheres) | | | | |
| Thickener | ACRYLAMIDE/SODIUM | 0.4 | 0.4 | 0.4 |
| | ACRYLOYLDIMETHYL- | | | |
| | TAURATE COPOLYMER | | | |
| Solvent | ISOHEXADECANE | 0.2 | 0.2 | 0.2 |
| Nonionic | LAURETH-21, | 0.1 | 0.1 | 0.1 |
| Surfactant | POLYSORBATE 80, & | | | |
| | SORBITAN OLEATE | | | |
| pH adjuster | DIMTHEYL MEA | | 0.4 | |
| Preservative | PRESERVATIVES | | ≤1 | |
| Water | WATER | QS | QS | QS |
| | | 100 | 100 | 100 |

Each composition was deposited on a synthetic leather substrate and allowed to dry at room temperature for 1 hour. The deposited compositions were then evaluated by experts. The results are presented in the table below.

| | J | C-1 | C-2 |
|---|---|---|---|
| Results | Transparent, | White, | Film is translucent |
| | uniform film, | cracked film | and white with white |
| | stretchable | | particles, can be |
| | without | | stretched without |
| | cracking | | cracking, but whitens |
| | | | upon stretching |

The foregoing description illustrates and describes the disclosure. Additionally, the disclosure shows and describes only the preferred embodiments but, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein. Also, it is intended to the appended claims be construed to include alternative embodiments.

As used herein, the terms “comprising,” “having,” and “including” are used in their open, non-limiting sense.

The terms “a,” “an,” and “the” are understood to encompass the plural as well as the singular. Thus, the term “a mixture thereof” also relates to “mixtures thereof.” Throughout the disclosure, the term “a mixture thereof” is used, following a list of elements as shown in the following example where letters A-F represent the elements: “one or more elements selected from the group consisting of A, B, C, D, E, F, and a mixture thereof.” The term, “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.”

Likewise, the term “a salt thereof” also relates to “salts thereof.” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be include, or a mixture of any two of A, B, C, D, E, F, a salt of A, a salt of B, a salt of C, a salt of D, a salt of E, and a salt of F may be included.

The expression “one or more” means “at least one” and thus includes individual components as well as mixtures/combinations.

Other than in the operating examples, or where otherwise indicated, all numbers expressing quantities of ingredients and/or reaction conditions may be modified in all instances by the term “about,” meaning within +/−5% of the indicated number.

Some of the various categories of components identified may overlap. In such cases where overlap may exist and the composition includes both components (or the composition includes more than two components that overlap), an overlapping compound does not represent more than one component. For example, a fatty acid may be considered both an emulsifier and a fatty compound. If a particular composition includes both an emulsifier and a fatty compound, a single fatty acid will serve as only the emulsifier or only the fatty compound (the single fatty acid does not serve as both the emulsifier and the fatty component).

The salts referred to throughout the disclosure may include salts having a counter-ion such as an alkali metal, alkaline earth metal, or ammonium counter-ion. This list of counter-ions, however, is non-limiting.

The term “treat” (and its grammatical variations) as used herein refers to the application of the compositions of the present disclosure onto the surface of keratinous substrates such as skin, in particular, the skin of the head, face, and neck.

As used herein, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc. All ranges and values disclosed herein are inclusive and combinable. For examples, any value or point described herein that falls within a range described herein can serve as a minimum or maximum value to derive a sub-range, etc.

The term “substantially free” or “essentially free” as used herein means that there is less than about 2% by weight of a specific material added to a composition, based on the total weight of the compositions. Nonetheless, the compositions may include less than about 1 wt. %, less than about 0.5 wt. %, less than about 0.1 wt. %, or none of the specified material. All of the components set forth herein may be optionally included or excluded from the compositions/method/kits. When excluded, the compositions/methods/kits may be free or essentially free of the component. For example, a particular composition may be free or essentially free of alkoxylated compounds, for example, ethoxylated thickeners and/or ethoxylated surfactants. Likewise, a particular composition may be free or essentially free of sulfates, such as sulfate surfactants.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

What is claimed is:

1. A cosmetic composition comprising:
about 1 to about 40 wt. % of at least one primary water-dispersible film forming polymer having a Young's modulus of 0.1 to 50 MPa, and elongation at break >50% and a glass transition temperature of <0° C.;
optionally, about 1 to about 35 wt. % of at least one secondary, non-polyurethane water-dispersible film forming polymer having a glass transition temperature of <25° C.,
optionally, about 1 to about 35 wt. % of at least one pressure sensitive adhesive polymer;
about 1 to about 35 wt. % of soft focus powder;
at least one thickening agent;
optionally, about 0.01 to about 25 wt. % of at least one water soluble solvent; and
water,
wherein the weight percentages are based on the total weight of the cosmetic composition.

2. The cosmetic composition of claim 1, wherein the glass transition temperature of the primary fim forming polymer(s) and the secondary film forming polymer(s), if present, differs by at least 20° C.

3. The cosmetic composition of claim 1, wherein the total amount of all film forming polymers in the composition is at least 10 wt. %, based on the total weight of the cosmetic composition.

4. The cosmetic composition of claim 1, wherein the weight ratio of the total amount of all primary water-dispersible film forming polymers to the total amount of all secondary film forming polymers is at least 2:1.

5. The composition of claim 1, wherein the at least one primary water-dispersible film forming polymer is a polyurethane latex polymer.

6. The composition of claim 5, wherein the at least one polyurethane latex polymer is selected from the group consisting of polycarbonate polyurethane, aliphatic polyurethane, aliphatic polyester polyurethane, Polyurethane 32, polyurethane-34, polyurethane-35, polyurethane-48, and mixtures thereof.

7. The composition of claim 6 comprising polyurethane-34 and/or polyurethane-35.

8. The cosmetic composition of claim 1, wherein the at least one optional secondary, non-polyurethane water-dispersible film forming polymer is an acrylate polymer.

9. The cosmetic composition of claim 8, wherein the acrylate polymer is selected from the group consisting of acrylates copolymer, styrene/acrylates copolymer, acarylates/ethylhexyl acrylate copolymer, an alkyl acrylate copolymer, acrylic copolymers, polyacrylate-2 crosspolymer, acrylates/hydroxyesters acrylate copolymer, acrylate/ethylhexyl acrylate copolymer, styrene acrylate copolymer, acrylate/VA copolymer, styrene/acrylic copolymer, styrene/acrylates copolymer, styrene/acrylates/ammonium methacrylate copolymer, and a mixture thereof.

10. The cosmetic composition of claim 9, wherein the acrylate polymer is acrylates copolymer.

11. The cosmetic composition of claim 1 comprising the at least one pressure sensitive adhesive polymer, and the at least one pressure sensitive adhesive polymer is a pressure sensitive adhesive acrylate polymer.

12. The cosmetic composition of claim 1 comprising the at least one pressure sensitive adhesive polymer, and the pressure sensitive adhesive polymer is a copolymer of butyl acrylate, butyl methacrylate or acrylic acid.

13. The cosmetic composition of claim 12, wherein the pressure sensitive adhesive acrylate polymer is a polymer of 2-ethylhexylacrylate.

14. The cosmetic composition of claim 1, wherein the soft focus powders are selected from the group consisting of talc, mica, titanated mica, alumina, aluminum silicate, silica which may or may not be coated, fumed silica, silica silylate, polyamide, poly(methyl (meth)acrylate), polyethylene powder, polymethylsilsesquioxane powder, copernicia cerifera (carnuauba) wax, nylon-12, cellulose, polylactic acid, boron nitride and a mixture thereof.

15. The cosmetic composition of claim 14 comprising silica which may or may not be coated and polymethylsilsesquioxane powder.

16. The cosmetic composition of claim 1, wherein the total amount of soft focus powders in the composition is at least 2 wt. %, based on the total weight of the cosmetic composition.

17. The cosmetic composition of claim 1, wherein the weight ratio of the total amount of all film forming polymers to the total amount of soft focus powders in the cosmetic composition is from about 1:1 to about 6:1.

18. The cosmetic composition of claim 1, wherein the at least one thickening agent is a hydrophilic thickening agent selected from the group consisting of modified or unmodified carboxyvinyl polymers, acrylates/C10-30 alkyl acrylate crosspolymer, polyacrylates, polymethacrylates, polyacrylamides, optionally crosslinked and/or neutralized 2-acrylamido-2-methylpropane sulfonic acid polymers, crosslinked anionic copolymers of acrylamide and of 2-acrylamido-2-methylpropane sulfonic acid (AMPS) (e.g., acrylamide/sodium acryloyldimethyltaurate copolymer), polysaccharides, celluloses, acrylamide/sodium acryloyldimethyl taurate copolymer, polyacrylate crosspolymer-6, sodium acrylates copolymer, hydroxyethylcellulose, acrylates/C10-30 alkyl acrylate crosspolymer, acrylates copolymer, sodium acrylates copolymer, hydroxypropyl distarch phosphate, steareth-100/peg-136/hdi copolymer, acrylates/steareth-20 methacrylate copolymer, and a mixture thereof.

19. The cosmetic composition of claim 18, wherein the at least one thickening agent is acrylamide/sodium acryloyldimethyltaurate copolymer.

20. The cosmetic composition of claim 1 comprising at least one water-soluble solvent selected from the group consisting of glycerin, an alcohol, an organic solvent, a polyols, a glycol, and a mixture thereof.

21. The cosmetic composition of claim 20 comprising ethanol.

22. The cosmetic composition of claim 1 comprising about 30 to about 90 wt. % of water.

23. A cosmetic composition comprising:
about 5 to about 40 wt. % of a first primary film forming polymer that is polyurethane-34
optionally, about 1 to about 20 wt. % of a second primary film forming polymer that is polyurethane-35;
optionally, about 1 to about 20 wt. % of a secondary film forming polymer that is acrylates copolymer;
optionally, about 1 to about 15 wt. % of a pressure sensitive adhesive polymer of 2-ethylhexylacrylate;
optionally, about 1 to about 30 wt. % of soft focus powder;
about 0.01 to about 5 wt. % of at least one hydrophilic thickening agent;
optionally, about 0.01 to about 25 wt. % of at least one water soluble solvent; and
about 30 to about 70 wt. % of water; and
wherein the weight percentages are based on the total weight of the cosmetic composition; and wherein the total amount of all film forming polymers in the composition is at least 10 wt. %, based on the total weight of the cosmetic composition.

25. A method for improving the appearance of skin comprising applying a cosmetic composition of claim 1 to the skin.

26. The method of claim 25, wherein improving the appearance of the skin comprises reducing dryness, roughness, dullness, age spots, sagging, eye bags, and/or reducing the appearance of wrinkles, blemishes, and/or other skin imperfections including scars.
